(19) United States
(12) Patent Application Publication
(10) Pub. No.: US 2013/0218278 A1
(43) Pub. Date: Aug. 22, 2013

(54) DEVICE AND METHOD FOR PERFORMING SPINAL INTERBODY FUSION

(71) Applicants: Steve Wolfe, Woodbury, MN (US); Clint Boylan, Minneapolis, MN (US)

(72) Inventors: Steve Wolfe, Woodbury, MN (US); Clint Boylan, Minneapolis, MN (US)

(21) Appl. No.: 13/773,554

(22) Filed: Feb. 21, 2013

Related U.S. Application Data
(60) Provisional application No. 61/601,488, filed on Feb. 21, 2012.

(51) Int. Cl. A61F 2/44

(52) U.S. Cl. CPC A61F 2/4455
USPC 623/17.16

(57) ABSTRACT

A system, device and method for performing a spinal interbody fusion is disclosed. The spinal fusion device may include a container that is placed in a prepared intervertebral cavity and then filled with a plurality of individual pieces or beads of PEEK or other biocompatible non-biologic material to provide mechanical support between the adjacent vertebra, and to distract the adjacent vertebrae and promote fusion.

### PRIORITY

This application claims the benefit of U.S. Provisional Application Ser. No. 61/601,488, filed on Feb. 21, 2012, which is hereby incorporated by reference in its entirety herein.

### FIELD

The present invention relates generally to a system, device and method for performing a spinal interbody fusion.

### BACKGROUND

It is recognized that the spinal disc consists of three parts: first, the nucleus, a central portion that is a compression-resisting cushion; second, the annulus, a peripheral rim portion that is a tension-resisting hoop; and third, the end plates, the superior and inferior borders of the disc, consisting of the upper and lower surfaces of the vertebral body bones adjacent to the disc. Many studies have concluded that mechanical back pain is the most common and costly musculoskeletal condition affecting middle-aged humans in modern societies. Mechanical back pain may be caused by several factors, but overwhelming evidence suggests that degeneration of the spinal intervertebral disc, such as may be caused by Degenerative Disc Disease (DDD) is the most common condition causing back pain symptoms.

Many devices have been invented for the purpose of stabilizing and/or replacing parts of the disc in an effort to ease the pain associated with degenerative disc disease. Previous devices designed to treat DDD fall generally into the following four classes:

The first class includes rigid, three-dimensional geometric solid devices, either impervious or porous, that function as support struts. When these devices are placed between adjacent vertebral bodies they allow, and in some cases encourage bone to grow through and/or around the device to cause a bony fusion between two adjacent vertebral bodies. Rigid implants fabricated from metal, ceramic, or hard plastics suffer from several disadvantages such as: the need to create large surgical exposures disruptive to muscle and soft tissue, the need for large destabilizing entrance holes through the annulus of the disc, and the presence of large volumes of non-biologic material that reduce bone graft surface contact at the end plate.

The second class involves the use of semi-rigid artificial joints that allow motion in one or more planes. Examples include: U.S. Pat. Nos. 4,759,769 to Kostuik; 6,039,763 to Shelokov, and commercially available examples such as the Link device or the Charite Intervertebral Disc Endoprosthesis. These artificial joints have several disadvantages, including: the artificial joints are technically challenging to the surgeon in that proper placement of the device can be quite difficult, placement of the device requires large anterior exposures and re-operation procedures, if needed, are dangerous (life-threatening) due to anterior scarring and inability to use another surgical approach.

The third class is directed to non-rigid cushions designed to replace the nucleus of the disc. Examples of artificial discs are described in U.S. Pat. No. 4,904,260 to Ray, U.S. Pat. No. 4,772,287 to Ray and U.S. Pat. No. 5,192,326 to Bao. These devices are prone to wear and subsidence and as such pose a risk to the surrounding anatomy when they become dislocated out of the disc space.

Finally, the fourth class is the relatively new area of initially flexible, deployable containers that become rigid when injected with materials that can support loads. Examples include U.S. Pat. Nos. 5,571,189, 5,549,679 and 6,712,853 to Kuslich, and co-pending application Ser. No. 12/650,889, the contents of each of which are incorporated in their entirety herein. Each of these describe deployable, porous containers, useful in stabilizing a deteriorating spinal disc. The container is placed into a reamed out intervertebral space and is expanded by the introduction of graft material which may be tightly compacted within the container.

Like many other areas of surgery, spine surgery has become less invasive as smaller, more precise technology develops. Many minimally invasive intervertebral fusion devices exist, such as those disclosed in U.S. Pat. Nos. 5,571,189 and 5,549,679 and the commercially available XLIF® procedure by NuVasive. However, all minimally invasive fusion devices still require a surgical access opening that is as large as the device to be implanted. Generally speaking, the access aperture in minimally invasive procedures is at least 15-30 mm in diameter. Also, because minimally invasive procedures require direct visualization, the surgeon may need to cut bone and must significantly retract soft tissues and the nerve root, potentially causing nerve root injury or persistent post-operative pain.

Any device that would more easily, and/or more effectively, and/or more safely treat degenerative disc disease would be useful in the management of hundreds of thousands of suffering individuals.

The entire content of each and all patents, patent applications, articles and additional references, mentioned herein, are respectively incorporated herein by reference.

The art described in this section is not intended to constitute an admission that any patent, publication or other information referred to herein is “prior art” with respect to this invention, unless specifically designated as such. In addition, this section should not be construed to mean that a search has been made or that no other pertinent information as defined in 37 C.F.R. §1.56 exists.

### SUMMARY

Systems, devices and methods for performing a spinal interbody fusion are disclosed. In certain embodiments, the spinal fusion device may include a container that is placed in a prepared intervertebral cavity and then filled with polyether ether ketone (PEEK) or other biocompatible beads and/or spheres to distract the adjacent vertebrae and promote fusion.

Certain embodiments of the present invention provide a device for performing spinal fusion on a prepared intervertebral cavity between two adjacent vertebra. The device comprises a container sized to be inserted through a less than 10 millimeter access opening; and a fill pack including a plurality of individual pieces of non-biologic material. The container is configured to contain the fill pack within the intervertebral cavity. The individual pieces of non-biologic material are constructed and arranged in the fill pack to provide mechanical support between the adjacent vertebra.

Additional embodiments of the present invention provide a device for performing spinal fusion on a prepared intervertebral cavity between two adjacent vertebra. The device comprises a container sized to be inserted through a less than 10 millimeter access opening; and a fill pack including a plurality of individual pieces of non-biologic material. The container is configured to contain the fill pack within the intervertebral cavity. The individual pieces of non-biologic material are constructed and arranged in the fill pack provide a bone growth scaffold between the adjacent vertebra.

Further embodiments of the present invention provide a method for performing spinal fusion on a prepared intervertebral cavity between two adjacent vertebra. The method comprises inserting a container through a less than 10 millimeter access opening into the prepared vertebral cavity between two adjacent vertebra and filling the container with a plurality of individual pieces of non-biologic material to form a fill pack configured to withstanding normal anatomic loads.

And further embodiments of the present invention provide a system for performing spinal fusion on a prepared intervertebral cavity between two adjacent vertebra. The system comprises a container sized to be inserted through a less than 10 millimeter access opening in the prepared vertebral cavity between two adjacent vertebra. The system also includes a plurality of pieces of non-biologic fill material configured to fill the container to form a fill pack contained within the container capable of withstanding normal anatomic loads. The system also includes a set of instructions for using the system.

In additional embodiments and aspects, the container can be porous. The container can also comprise knitted PEEK thread. Bone growth promoting biologic fill material can also be placed outside of the container to promote bone growth.

In additional embodiments and aspects, the fill pack can include bone growth promoting biologic fill material. The individual pieces of non-biologic material can each comprise PEEK. The individual pieces of non-biologic material can each be bead-shaped. Also, the individual pieces of non-biologic material can comprise a radiolucent material.

It should be understood, however, that the intention is not to limit the invention to the particular example embodiments described. On the contrary, the invention is to cover all modifications, equivalents, and alternatives falling within the spirit and scope of the invention as defined by the appended claims.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a container filled with beads according to various embodiments of the present invention.

FIG. 2 depicts a perspective view of a device according to various embodiments of the present invention, placed between two vertebrae.

### DETAILED DESCRIPTION

In the following descriptions, the present invention will be explained with reference to example embodiments thereof. However, these example embodiments are not intended to limit the present invention to any specific examples, embodiments, environments, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

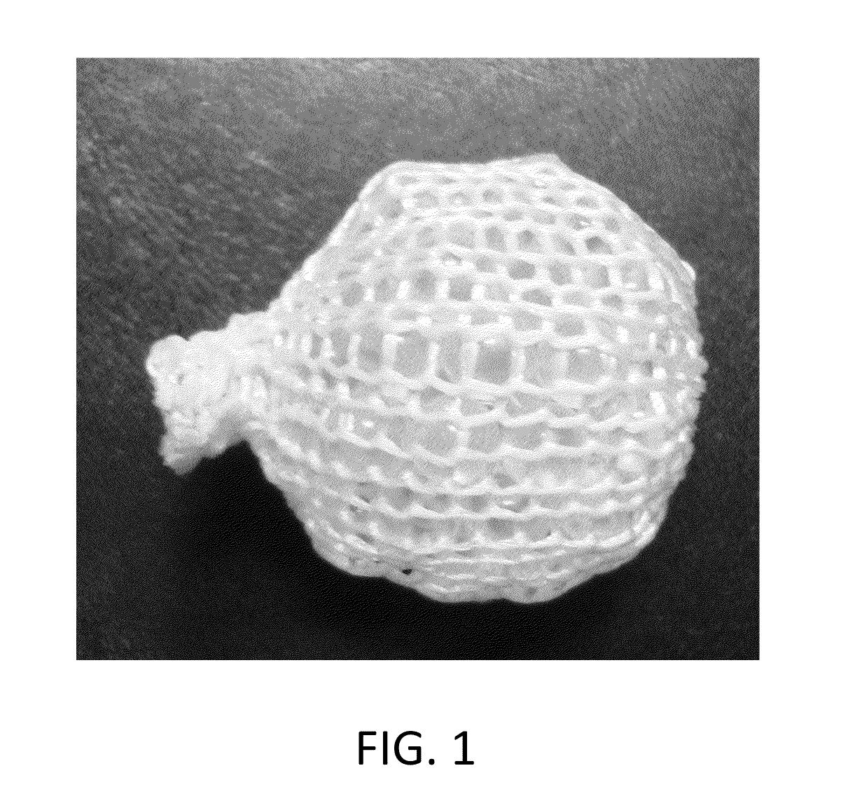

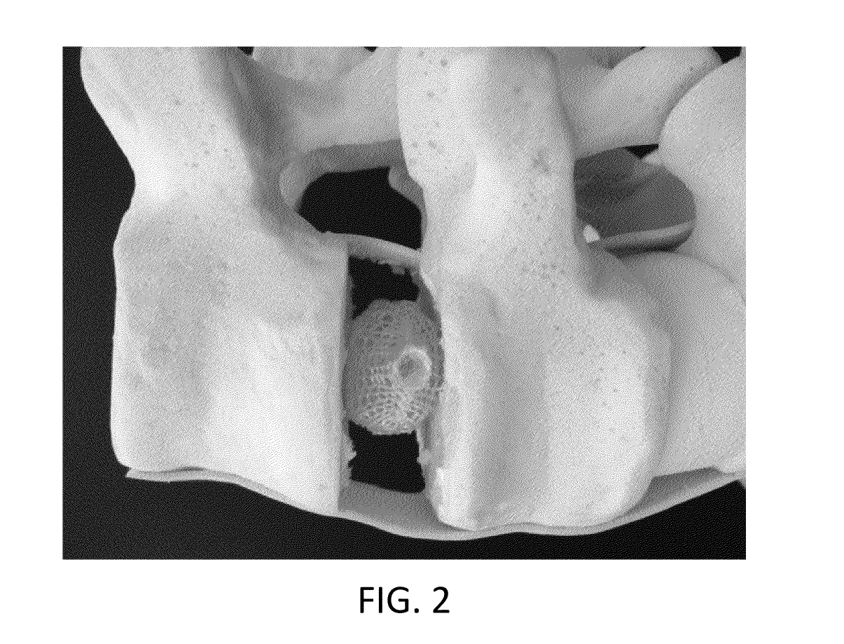

The spinal fusion device according to an embodiment of the present invention includes an implant for intervertebral bony fusion that is constructed in-situ through an access opening in the patient's anatomy that is less than 10 mm. Referring to FIGS. 1 and 2, the implant may include a container that is placed in a prepared intervertebral cavity and then filled with PEEK or other non-biologic biocompatible pieces to distract the adjacent vertebrae and promote fusion. Such preparation of the intervertebral cavity may include performing a discectomy.

The container according to an example embodiment is disclosed in U.S. Patent Nos. 5,549,679 and 5,571,189, the contents of which are incorporated in their entirety herein, such that the container is constructed from material, such as PET or PEEK, that is woven, knitted, braided or form-molded to a density that will allow ingress and egress of fluids and solutions and will allow the ingrowth and through-growth of blood vessels and fibrous tissue and bony trabeculae to promote fusion, but the porosity may be tight enough to retain small particles of enclosed material.

The container can include a plurality of pores. Generally, the pores have a diameter of about 0.25 mm or less to about 5.0 mm. The size is selected to allow tissue ingrowth and bony fusion while containing the material packed into the container. When the container is fully filled with fill material, the container will form a self-retaining shape which conforms to and substantially fills the cavity.

The size and density of the pores determine the ease or difficulty with which materials may pass through the container. For instance, very small pores (<0.5 mm) would prohibit passage of all but the smallest particles and liquids. The pore size and density can be controlled in the manufacturing process, such that the final product is matched to the needs of the surgeon.

The container need not be woven and may be molded or otherwise formed as is known in the art. The preferred material provides the ability to tailor bioabsorbance rates, for example, such as is disclosed in co-pending application Ser. No. 11/901,237, the disclosure of which is incorporated by reference herein in its entirety. However, any suture-type material used medically may be used to form the container. The container can also be formed of plastic, metal or a combination of multiple materials. In at least one embodiment, container is formed of a combination of resorbable and/or nonresorbable thread. The container can be partially or totally absorbable, metal, plastic, woven, solid, film, PEEK, an extruded balloon or any other biocompatible material.

The container according to various embodiments can be radio-opaque or include markings for x-ray visualization during insertion and filling. In an embodiment, such marking includes pad printing or other marking method with a biocompatible ink. According to one aspect, medical grade biocompatible radio-opaque ink may be loaded with tantalum powder. Marking may be placed at any desired location. In a preferred embodiment, markings are placed at the proximal and distal ends of the container.

The amount of fill material required to create the appropriate fill pack to fill a container can be determined by the container size and desired final shape. Fill materials and tools for inserting the fill materials according to various embodiments of the present invention have been disclosed in the following patents and co-pending U.S. Pat. Nos. 6,620,169, 6,620,162, 7,025,771 and Ser. No. 10/924,240, the disclosures of which are incorporated in their entirety herein.

In a preferred embodiment, the container is filled in situ with fill material comprising PEEK beads and/or PEEK pieces, or other non-biologic biocompatible material such as polymers, metals (e.g. titanium), ceramics, or a combination thereof. The pieces can be shaped as beads, spheres, tubes, cylinders or any other desired shape to form a fill pack within the container capable of withstanding normal anatomic loads and to provide mechanical structural support between the vertebra, similar to the mechanical support provided by a traditional block of PEEK used as a fusion implant. But unlike a traditional PEEK block, the pack formed of a plurality of individual pieces defines a plurality of interstitial spaces. The resulting combination of pieces and spaces defines a scaffold for bone growth having controllable porosity such that osteointegration may be optimized.

The individual pieces can be sized in the range from about 0.5 mm to 5 mm in diameter. Multiple different bead sizes and/or shapes can be used to fill a given container. Alternatively, a single uniform sized and shaped bead can be used to fill the container. In an example embodiment, larger big beads may be used first to fill out the container and then small beads that behave more fluidly may be introduced to complete the filling operation and distract adjacent vertebra.

The shape of the pieces/beads may be varied to control the stiffness and/or mechanical properties of the fill material packed inside the porous container. For example, PEEK tubing may be cut to specific lengths and stacked to achieve a desired stiffness of the pack. Notches may cut into the PEEK pieces to such that the fill material pack may be more easily compressed.

The container fill passage can be closed in a variety of ways to ensure that beads do not migrate out of the container. For example, but not limited to: the container may be tied off, a plug may be disposed in the opening to seal the opening, elastic cinches may be knit into the neck of the container, an elongated neck may be tucked inside out such that the neck folds in on itself and/or the neck may fold over on outside the outside of the container and clamp shut. Any other suitable method of closing the container may also be used.

In an embodiment, the fill material also includes bone graft or other biologic or biocompatible materials known to promote bone growth and/or fusion. In another embodiment, bone graft or other biologic or biocompatible material known to promote bone growth and/or fusion may be placed around the container in the disc space as in a sentinel graft.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A device for performing spinal fusion on a prepared intervertebral cavity between two adjacent vertebra, comprising:
a container sized to be inserted through a less than 10 millimeter access opening; and
a fill pack including a plurality of individual pieces of non-biologic material,
wherein the container is configured to contain the fill pack within the intervertebral cavity, and
wherein the individual pieces of non-biologic material are constructed and arranged in the fill pack to provide mechanical support between the adjacent vertebra.

2. The device according to claim 1 wherein the container is porous.

3. The device according to claim 1 wherein the fill pack further includes bone growth promoting biologic fill material.

4. The device of claim 1, wherein the individual pieces of non-biologic material each comprise PEEK.

5. The device of claim 4, wherein the individual pieces of non-biologic material are each bead-shaped.

6. A device for performing spinal fusion on a prepared intervertebral cavity between two adjacent vertebra, the device comprising:
a container sized to be inserted through a less than 10 millimeter access opening; and
a fill pack including a plurality of individual pieces of non-biologic material,
wherein the container is configured to contain the fill pack within the intervertebral cavity, and
wherein the individual pieces of non-biologic material are constructed and arranged in the fill pack provide a bone growth scaffold between the adjacent vertebra.

7. The device according to claim 6 wherein the container is porous.

8. The device according to claim 6, wherein the container comprises knitted PEEK thread.

9. The device of claim 6, wherein each of the pieces of non-biologic material comprise PEEK.

10. The device of claim 6, wherein each of the pieces of non-biologic material are bead-shaped.

11. The device of claim 6, wherein the fill pack includes bone growth promoting biologic fill material.

12. A method for performing spinal fusion on a prepared intervertebral cavity between two adjacent vertebra, comprising:
inserting a container through a less than 10 millimeter access opening into the prepared vertebral cavity between two adjacent vertebra;
filling the container with a plurality of individual pieces of non-biologic material to form a fill pack configured to withstanding normal anatomic loads.

13. The method of claim 13, further including adding bone growth promoting biologic fill material to the fill pack.

14. The method of claim 13, further including placing bone growth promoting biologic fill material outside of the container to promote bone growth.

15. A system for performing spinal fusion on a prepared intervertebral cavity between two adjacent vertebra, comprising:
a container sized to be inserted through a less than 10 millimeter access opening in the prepared vertebral cavity between two adjacent vertebra;
a plurality of pieces of non-biologic fill material configured to fill the container to form a fill pack contained within the container capable of withstanding normal anatomic loads;
a set of instructions for using the system.

16. The system of claim 15, further including providing bone growth promoting biologic fill material.

17. The system of claim 15 wherein the non-biologic material is PEEK.

18. The system of claim 15 wherein the individual pieces of non-biologic material comprise a radiolucent material.

19. The system of claim 15 wherein the pieces of non-biologic fill material are bead-shaped.

20. The system of claim 15 wherein the container comprises knitted PEEK thread.
